(19) United States
(12) Patent Application Publication
(10) Pub. No.: US 2024/0423534 A1
(43) Pub. Date: Dec. 26, 2024

(54) Treatment plan facilitator

(71) Applicant: NCASE INC, Las Vegas, NV (US)

(72) Inventors: Wesley P. WILSON, Athens, AL (US); Anthony OSIBOV, Las Vegas, NV (US)

(73) Assignee: NCASE INC, Las Vegas, NV (US)

(21) Appl. No.: 18/824,657

(22) Filed: Sep. 4, 2024

Related U.S. Application Data
(63) Continuation of application No. 17/052,661, filed on Nov. 3, 2020, now abandoned.
(60) Provisional application No. 62/667,419, filed on May 4, 2018.

(51) Int. Cl. A61B 5/00

(52) U.S. Cl. CPC A61B 5/4833; A61B 5/4547; A61B 5/4827

(57) ABSTRACT

A medical treatment tracking device with a cavity configured to store a medical device and one or more sensor configured to obtain data about the medical device. Based on the data obtained by the sensor, adherence to an aspect of a treatment plan is determined by one or more processor. The one or more processor is also configured to determine compliance with the treatment plan based on the adherence determination. A transmitter may then transmit the compliance determination may to an external device.

### CROSS-REFERENCE TO RELATED APPLICATION(S)

This patent application is a continuation of co-pending U.S. patent application Ser. No. 17/052,661, filed 2020 Nov. 3 which is a 371 of International Patent Application Serial No. PCT/US2019/030965, filed 2019 May 6, which claims priority to U.S. Provisional Patent Application Ser. No. 62/667,419, filed 2018 May 4, all of which are incorporated herein by reference.

### TECHNICAL FIELD

This patent application relates to the field of facilitating medical treatment plans. More specifically, this patent application relates to determining compliance with a medical treatment plan based on data corresponding to one or more aspect of the medical treatment plan.

### BACKGROUND

Medical practitioners are able to aid treatment with the use of medical devices. For some such devices, it is desirable that the patient is able to temporarily or permanently remove such device. For instance, a dentist or oral surgeon may provide an intraoral device or appliance to a patient for corrective or cosmetic treatment. For some treatments using such intraoral devices or appliances, it is preferred that the device or appliance be removable. If such a device or appliance is removed, it may be necessary to reapply or reinsert the device or appliance to continue treatment. Additionally, other aspects of the treatment process may need to be properly adhered to in order to adequately progress with the treatment process.

Although a medical practitioner may develop a treatment plan implementing a medical device, the patient may not accurately follow the treatment plan. This can lead to prolonged treatment time and/or the need to adjust the treatment plan to ensure favorable results. Accordingly, it may be desirable to track a patient's compliance to a treatment plan. It may also be desirable to track the patient's compliance without the need to rely on the patient's memory, the patient's honesty, and/or direct communication with the patient.

### SUMMARY

In one embodiment, a medical treatment tracking device may comprise a storage device having an opening for receiving a medical device. On or more sensor may be configured to detect whether or not the medical device is within the opening of the storage device. A storage device may be configured to store time data corresponding to a time the medical device is detected within the opening. A transmitter may be configured to transmit the time data.

In another embodiment, a dental appliance storage device may have a cavity configured to store a dental appliance. A storage device may be configured to store treatment plan data that includes one or more aspect of a treatment plan. One or more sensor may be configured to obtain data for determining adherence to one or more aspect of the treatment plan. One or more processor may be configured to determine adherence to one or more aspect of the treatment plan. The one or more processor may also be configured to determine compliance with the treatment plan based on the adherence determination(s). A transmitter may be configured to transmit compliance data to an external device, the compliance data corresponding to the compliance determination.

In another embodiment, a dental appliance storage device may have a cavity configured to store a dental appliance. One or more sensor may be configured to sense one or more aspect of the dental appliance. A transmitter may be configured to transmit at least a portion of the data corresponding to the sensed aspect(s) of the dental appliance.

In another embodiment, a method of determining compliance with a treatment plan may comprise the step of parsing one or more aspect of a treatment plan. Another step may include sensing one or more feature of a medical device corresponding to the one or more aspect of the treatment plan. Another step may include determining adherence to each aspect of the treatment plan based on the sensed feature(s) of the medical device. Another step may include determining compliance with the treatment plan based on the determination(s) of adherence of the aspect(s) of the treatment plan.

### BRIEF DESCRIPTION OF THE DRAWINGS

FIG. 1 depicts a perspective view of an embodiment of a dental appliance case.

FIG. 2 depicts a perspective view of an embodiment of a dental appliance case sensor array.

FIG. 3 depicts a perspective view of an embodiment of circuitry including a transmitter.

FIG. 4 depicts a bird's eye view of an embodiment of circuitry including a sensor array.

FIG. 5 illustrates a block diagram of an embodiment of some of the components of a dental appliance case.

FIG. 6 shows a block diagram of an embodiment of some of the components of an external client.

FIG. 7 depicts a perspective view of an embodiment of a dental appliance case in wireless communication with external client devices.

FIG. 8 depicts a perspective view of an embodiment of an external server in wireless communication with an external client device and a dental appliance case.

FIG. 9 shows a block diagram of an embodiment of an external server.

FIG. 10 depicts perspective view of an embodiment of an external client synchronously syncing data between two dental appliance cases.

### DETAILED DESCRIPTION

The following detailed description is provided to assist the reader in gaining a comprehensive understanding of the methods, apparatuses, and/or systems described herein. However, various changes, modifications, and equivalents of the methods, apparatuses, and/or systems described herein will be apparent after an understanding of the disclosure of this application. For example, the sequences of operations described herein are merely examples, and are not limited to those set forth herein, but may be changed as will be apparent after an understanding of the disclosure of this application, with the exception of operations necessarily occurring in a certain order. Also, descriptions of features that are known in the art may be omitted for increased clarity and conciseness.

The features described herein may be embodied in different forms, and are not to be construed as being limited to the examples described herein. Rather, the examples described herein have been provided merely to illustrate some of the many possible ways of implementing the methods, apparatuses, and/or systems described herein that will be apparent after an understanding of the disclosure of this application.

Terms such as first, second, A, B, (a), (b), and the like may be used herein to describe components. Each of these terminologies is not used to define an essence, order, or sequence of a corresponding component but used merely to distinguish the corresponding component from other component(s). For example, a first component may be referred to a second component, and similarly the second component may also be referred to as the first component.

It should be noted that if it is described in the specification that one component is “connected,” “coupled,” or “joined” to another component, a third component may be “connected,” “coupled,” and “joined” between the first and second components, although the first component may be directly connected, coupled, or joined to the second component. In addition, it should be noted that if it is described in the specification that one component is “directly connected” or “directly joined” to another component, a third component may not be present therebetween. Likewise, expressions, for example, “between” and “immediately between” and “adjacent to” and “immediately adjacent to” may also be construed as described in the foregoing.

The terminology used herein is for the purpose of describing particular embodiments only and is not intended to be limiting. As used herein, the singular forms “a,” “an,” and “the,” are intended to include the plural forms as well, unless the context clearly indicates otherwise. It will be further understood that the terms “comprises,” “comprising,” “includes,” and/or “including,” when used herein, specify the presence of stated features, integers, operations, elements, and/or components, but do not preclude the presence or addition of one or more other features, integers, operations, elements, components, and/or groups thereof.

Unless otherwise defined, all terms, including technical and scientific terms, used herein have the same meaning as commonly understood by one of ordinary skill in the art to which this disclosure pertains based on an understanding of the present disclosure. Terms, such as those defined in commonly used dictionaries, are to be interpreted as having a meaning that is consistent with their meaning in the context of the relevant art and the present disclosure, and are not to be interpreted in an idealized or overly formal sense unless expressly so defined herein.

A common increasing trend in the intraoral industry is to provide corrective and cosmetic treatment by use of intraoral devices, such as dental appliances. One of the more popular treatments includes the use of fixed dental appliances for shifting and aligning a patient's teeth. Using traditional methods, such fixed dental appliances are adhered to one's teeth. However, as one's teeth are shifted by the dental appliance, the dental appliance may need to be readjusted to continue the treatment process. This often requires the use of physical force to break the adhesive bond between the tooth and the dental appliance. Further, the adhesive on the tooth may need to be removed before the adjusted appliance may be attached.

The dental industry is currently developing removable dental appliances that perform nearly the same function as fixed dental appliances. The dental industry is also using newer materials and techniques to improve the cosmetic appearance of the device while treatment is in progress.

However, with the use of such dental appliances, a dental practitioner must rely on a patient to remove and reinsert the dental appliance at appropriate time to ensure proper treatment according to the treatment plan. The dental practitioner must also rely on a patient to accurately follow various aspects of a dental plan. Unfortunately, a patient's strict compliance to a prescribed dental treatment plan may not always be present.

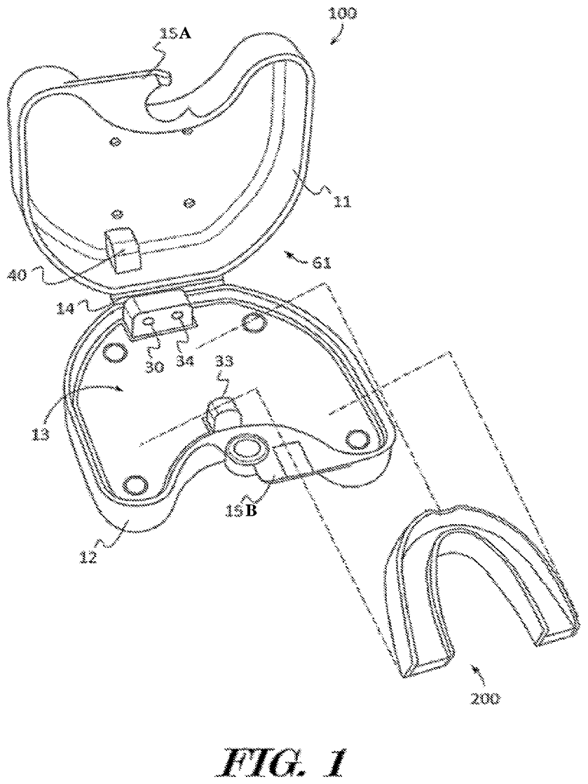

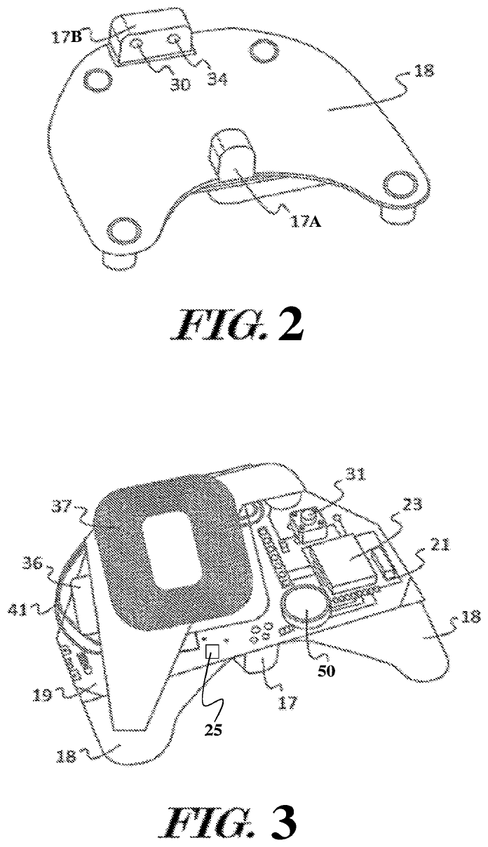

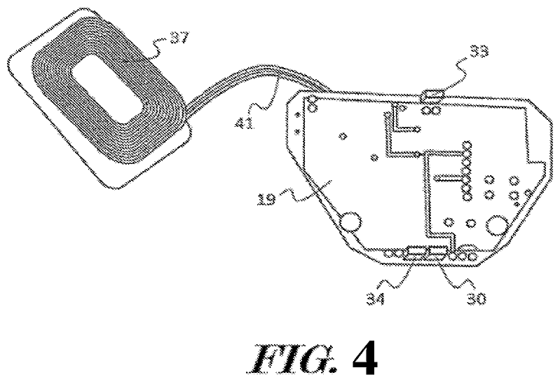

FIGS. 1-4 depict an embodiment of a medical device storage device, such as a dental appliance case 100, and embodiments of the components thereof.

In some embodiments, the dental appliance case 100 may comprise a lid 11 and a base 12 connected to each other by a hinge 14. The hinge 14 may connect and align the lid 11 and the base along one or more facets of the lid 11 and base 12. The lid may include a lid protrusion 40 that protrudes from one or more facets of the lid 11. FIG. 1 depicts an embodiment where the lid protrusion 40 protrudes from an inner-upper surface of the lid 11, and spaced apart from an inner-side surface of the lid 11. The lid 11 may further comprise a first lid release 15, which may assist with temporarily securing the lid 11 to the base 12.

A cavity 13 may be formed when the lid 11 and the base 12 are brought together. Various components may be positioned within the cavity 13. For instance, a dental appliance 200 may be at least partially positioned within the cavity 13. The positioning of the dental appliance 200 within the cavity 13 may server the function of storing and protecting the dental appliance 200.

A first sensor housing 17A and a second sensor housing 17B may be positioned within the cavity 13. The first sensor housing 17A and/or the second sensor housing 17B may house one or more sensors. In other embodiments, some of the components for the proper functionality of one or more sensor may be split across the first sensor housing 17A and the second sensor housing 17B. For instance, the first sensor housing 17A may house a sensor emitter 33 while the second sensor housing 17B may house a sensor receiver 34. In such an embodiment, both the sensor emitter 33 and sensor receiver 34 may be used in combination for sensing purposes. For instance, they may be used in combination as an occupancy sensor 32, which may be configured to sense the presence and/or absence of the dental appliance 200 within the cavity 13 of the dental appliance case 100. Other sensors may be used for obtaining other information about the dental appliance 200 and/or aspects of a dental treatment plan.

In some situations, the first sensor housing 17A and/or the second sensor housing 17B may house more than one sensor. For example, the second sensor housing 17B may house a sensor for sensing light, such as the sensor receiver 34, and may additionally house a lid sensor 30. The lid sensor 30 may be configured to sense the state of the lid 11, such as whether the lid 1 lis in an open or closed position. For instance, a lid sensor 30 may sense the proximity of the lid protrusion 40 to determine the state of the lid 11.

A floor 18 may be positioned within the cavity 13. The floor 18 may be positioned in such a fashion that sensitive electronics can be kept separate from the potentially contaminated area due to the presence and/or storage of the dental appliance 200. The floor 18 may also be positioned to separate various components located in the cavity 13.

For instance, a circuit board 19 may be positioned on one side of the floor 18, while the dental appliance is positioned on an opposite side. Various components may be physically and/or electrically connected to the circuit board 19. For example, the lid sensor 30, the sensor emitter 33, and the sensor receiver 34 may be connected to one or more sides of the circuit board 19. On the an opposite side, or on the same side, or a combination of the same side and opposite side, of the circuit board 19, a control input 31, a communication array 23, one or more processing unit 21, an alert device 50, memory 25, wires 41, a power source 36, and/or a charging receiver 37 may be electrically and/or physically connected to the circuit board 19.

In some embodiments, the alert device 50 may comprise one or more of a light emitting device 39, a sound device 36, and/or a vibrating device 38.

The base 12 of the dental appliance case 100 may include a second lid release 15B. The first lid release 15A and the second lid release 15B may be brought together and/or mated. In such a situation, the lid 11 and the base 12 may be temporarily aligned and secured together.

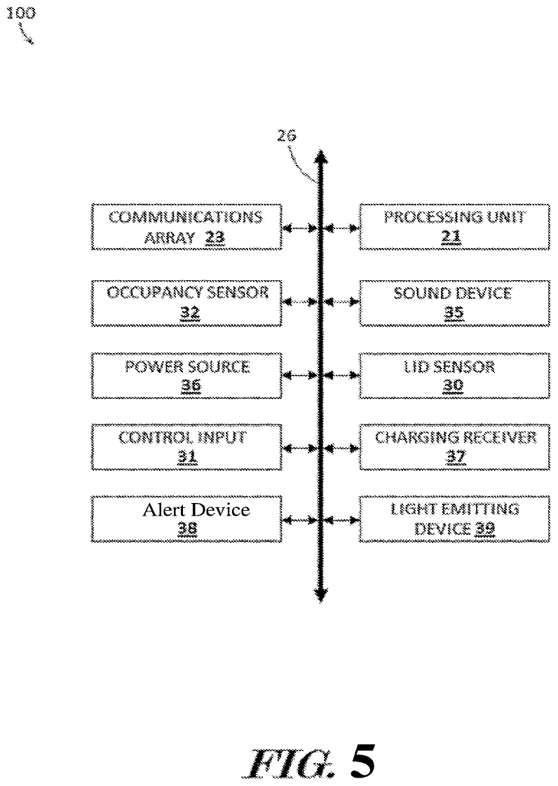

FIG. 5 illustrates a block diagram of an embodiment of some of the components of a dental appliance case. A common bus 26 electrically connects a communication array 23, occupancy sensor 32, power source 36, control input 31, vibrating device 38, processing unit 21, sound device 35, lid sensor 30, charging receiver 37, and light emitting device 39. Other components may optionally be connected to the common bus 26.

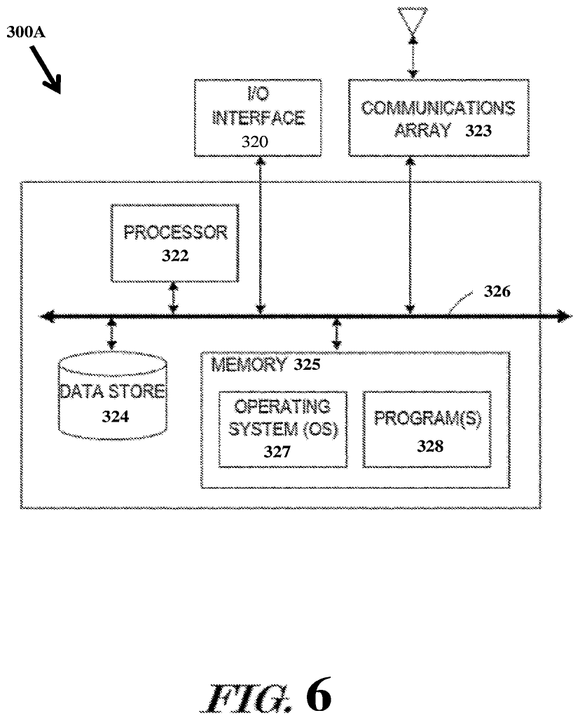

FIG. 6 shows a block diagram of an embodiment of some of the components of an external client, such as a mobile phone 300A. A common client bus 326 may electrically connect one or more client processor 322, client I/O interface 320, client communications array 323, client memory 325, client operating system 327, client program(s) 28, and/or client data store 324.

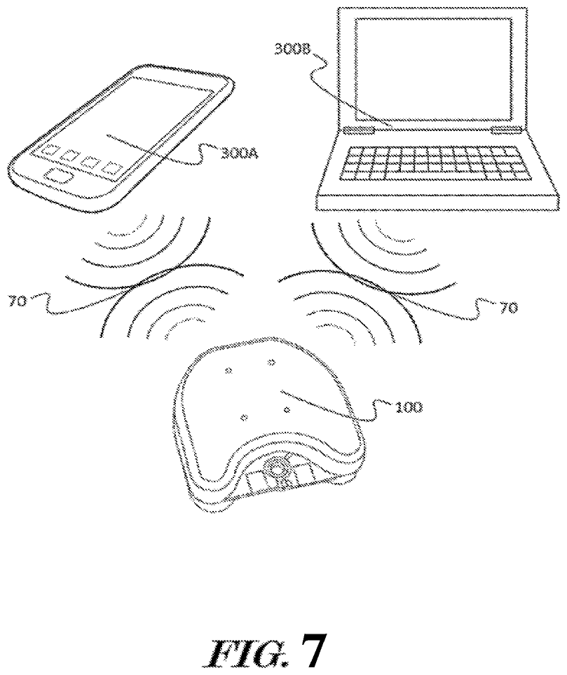

FIG. 7 depicts a perspective view of an embodiment of a dental appliance case in wireless communication with external client devices. The dental appliance case 100 may communicate, for example using the communication array 23, with one or more external client, the external client also optionally using a communication array. The external client can be any preferred external client, such as a smart phone 300A and/or a computer 300B. A communication link 70 may be wired or wireless.

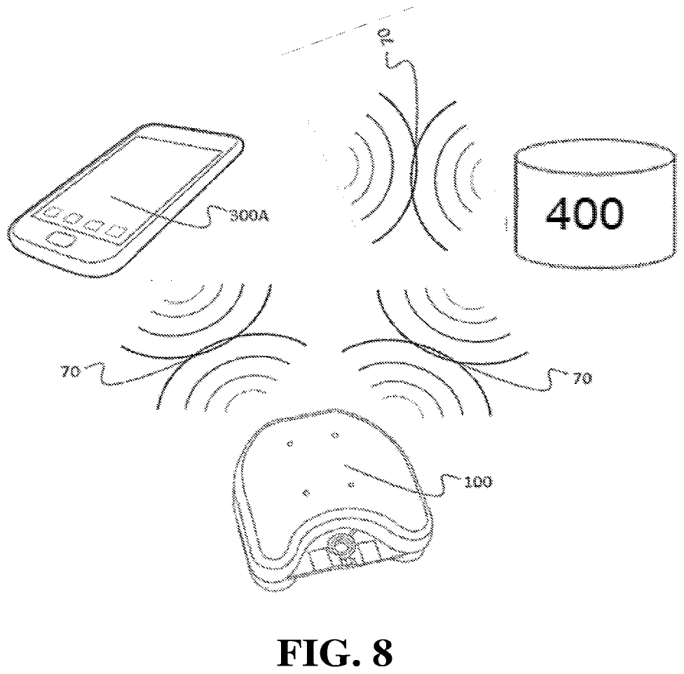

FIG. 8 depicts a perspective view of an example of an external server 400 in communication with a dental appliance case 100 and a client device, such as a smart phone 300A. The communication methods may be wired or wireless, employing such means as known in the art or any obvious variation thereof.

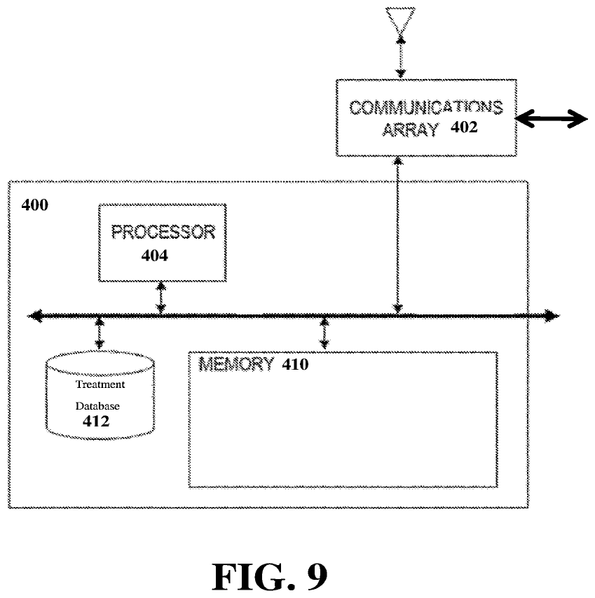

FIG. 9 shows a block diagram of an embodiment of an external server. The external server 400 may contain various components such as a server communication array 402, a server processor 404, a server memory 410, and/or a treatment database 412. The server memory 410 may store various types of information such as a treatment plan corresponding to the patient of the dental appliance 200. Alternatively or in addition to, the treatment plan may be stored in the treatment database 412.

The treatment plan corresponding to the patient of the dental appliance 200 may include various types of information related to a dental treatment procedure. For instance, the treatment plan may include information corresponding to the length of time the patient should wear the dental appliance 200 and/or the length of time modified, updated, or new dental appliance(s) should be worn or which dates such a modified, new, or updated dental appliance(s) should be worn. Other examples include information corresponding to the time of day and the geographical location such appliance(s) should be worn. As yet another example, the information may correspond to the timing and/or method that such appliance(s) should be adjusted. Still other examples include information corresponding to the movement of the dental appliance 200 and/or physical characteristics of the dental appliance 200. For instance, physical characteristic may include such characteristics as the cleanliness, physical structure, rigidity, damage, etc. of the dental appliance.

In some circumstances, compliance and its corresponding data may correspond to if the patient is appropriately adhering to certain aspects of a prescribed treatment plan, such aspects being based on the type of information related to the dental treatment procedure. For compliance purposes, adherence to aspects of the treatment plan may correspond to strict adherence or may correspond to adherence within a predetermined range. For instance, if a patient's treatment plan includes an aspect of wearing the dental appliance 200 for at least 20 hours a day, compliance may be deemed met if the dental appliance 200 has been worn for at least 20 hours a day, or within a predetermined number of minutes or hours of the prescribed 20 hours of wear a day.

Compliance may correspond to adherence to all aspects of the treatment plan, or may include adherence to only certain aspects of the treatment plan. Additionally, compliance may correspond to strict adherence of all relevant aspects of the treatment plan, adherence within a range of all relevant aspects of the treatment plan, or strict adherence of certain relevant aspects and less than strict adherence to other relevant aspects. As another example, compliance may be based on a weighted value of two or more relevant aspects of the treatment plan.

The compliance determination may be stored in the memory 25 of the case 100 and/or an external storage device, such as the server memory 410 and/or a storage device within the client, such as the client memory 325. A compliance determination may be performed and/or stored every hour, day, or other time. Further compliance determinations may be made based on the previously stored compliance determinations. For instance, a weekly compliance determination may be based on the daily compliance determinations for that week. Aggregate compliance may also be stored based on number of consecutive days of compliance or non-compliance. Other way of aggregating compliance determinations may also be possible.

Various ways of determining compliance may allow for certain flexibilities in the treatment plan. For example, a treatment plan for the month may be considered to be in compliance if each and every week of that month was determined to be in compliance. However, each week may be determined to be in compliance if a minimum of five days each week were determined to be compliant. In some situations, even if each week has been determined to be compliant, the month may be considered non-compliant if a certain number of consecutive days have been determined to be non-compliant, especially if such non-compliance is near the date that an updated treatment plan or dental appliance is to be provided to the patient.

The external server 400 may transmit, for example via the server communication array 402, one or more aspect of one or more treatment plan to the client, such as a smart phone 300A and/or computer 300B, and/or to the dental appliance case 100. Alternatively, the one or more aspect of the one or more treatment plan may be transmitted from the external server 400 to the dental appliance case 100 via the client. Upon receipt, the one or more aspect of the one or more treatment plan may be stored, for example in memory 25 of the dental appliance case 100.

The external server 400 may also transmit, for example via the server communication array 402, data corresponding to compliance to the client, such as a smart phone 300A and/or computer 300B, and/or to the dental appliance case 100. For instance, the compliance data may include the amount of adherence needed in order for the patient to be deemed in compliance with the prescribed treatment plan.

In some embodiments, the compliance data may be obtained directly from the dental appliance, or may obtain the compliance data from a combination of different sources, such as, for example, from a combination of the dental appliance and the external server 400. In one instance, the one or more sensor of the dental appliance case 100 or the smart phone 300A may be used to obtain the compliance data directly from the dental appliance 200. For example, the dental appliance may comprise an electronically readable tag or a physical code that contains data corresponding to the compliance data. The data may directly contain the compliance data, or may instruct the reading device to obtain some or all of the compliance data from an alternative source, for example from the external server 400. In some instances, obtaining the compliance data from one or more source may aid in securing the patient's sensitive information.

The dental appliance case 100 may use one or more sensor to determine an aspect of the dental appliance. For instance, one or more sensor may be configured to detect the presence and/or absence of the dental appliance 200. Other aspects of the dental appliance 200 that may be detected by one or more sensor include the structural integrity of the dental appliance 200, the cleanliness of the dental appliance 200, the current physical shape of the dental appliance 200, etc.

Based on data collected from the one or more sensor, one or more processing unit 21 may be used to determine various aspects of the dental appliance 200. For instance, the one or processing unit 21 may be configured to calculate the time that the dental appliance 200 was within the cavity 13 of the dental appliance case 100 based on the data collected from the one or more sensor. As an example, one or more processing unit 21 may access a timing element, for instance an internal clock, to determine the time that the dental appliance 200 is deemed to be located within the cavity 13 of the dental appliance case 100, such as based on the data received from the occupancy sensor 32. Such a determination may be based on, for instance, the comparison of the start time that the dental appliance 200 is deemed to be located within the cavity 13 of the dental appliance case 100, and the end time at the dental appliance case 200 is deemed to not be located within the cavity 13 of the dental appliance case 100. As another example, such determination may be based on a time duration, for instance based on a timer, that the dental appliance 200 is deemed to be continuously located within the cavity 13 the dental appliance case 100. As yet another example, the time duration that the dental appliance 200 is deemed to be located within the cavity 13 of the dental appliance case 100 may be based on a cumulative duration of time over a period of time, for instance one day or one month, and/or cumulative duration of time of all timers over a period of time. In one example, a plurality of timers may be located within a single dental appliance case 100 or spread out over numerous separate dental appliance cases.

Other examples of the aspects of the treatment plan that may be detected by the sensors include, but are not limited to, cleanliness, structural integrity, and patient specific information, such as teeth movement speed and pain thresholds. If cleanliness is an aspect of the treatment plan, sensors may be able to determine cleanliness by the amount absorption, transmission, and/or reflection of visible or non-visible light through the dental appliance 200. Other sensors may be used if cleanliness is determined in another fashion, such as by odor or chemical composition. If structural integrity is an aspect of the treatment plan, sensors may perform a visual inspection of the dental appliance 200, or may perform other inspections such as by acoustic analysis, radar reflection, light transmissivity, use time, etc.

In some embodiments, adherence to aspects of the dental plan may be determined based on data obtained from sensors located separate from the case 100. For instance, a sensor may include an optical sensor, such as a camera, located in the client, such as a smart phone 300A or computer 300B, or other external device. The optical data could be used to determine the structural integrity of the dental appliance 200 and/or determine if there is proper fit of the dental appliance within a patient's mouth, and/or progress of treatment, such as the movement of a patient's teeth.

In some embodiments, adherence to one or more aspect of the dental plan may be determined based on user input, such as by the patient, rather than based on, or in conjunction with, data from the one or more sensors. For instance, one aspect of the dental plan may be a perception of pain, which may indicate a patient's pain threshold. A patient may input a level of pain being experienced by the current dental appliance 200. If the perceived pain is above a certain threshold or below another threshold, an updated dental appliance and/or an updated dental plan may be provided. As another example, more than one dental appliance may be provided at a time. In such a situation, the patient may be able to select which dental appliance is more comfortable and if the pain caused by each dental appliance is bearable. This may be beneficial as patients with higher pain thresholds may be able to speed up treatment or a certain level of pain may indicate that the fit of the dental appliance is not proper.

As another example, the user, such as the patient, may input data corresponding to adherence to an aspect of the dental plan that could otherwise potentially be obtained from sensors. For instance, a user may be able to provide information corresponding to whether the structural integrity of the dental appliance is adequate. The user may also be able to correct or modify data or determinations of adherence to aspects of the dental plan. For instance, the user may modify the time that the dental appliance 200 was not being worn, despite the dental appliance not being detected within the cavity 13 of the case 100.

Based on the data collected on an aspect of the dental appliance 200, the one or more processor 21 may be used to determine adherence to a corresponding aspect of the dental plan. The one or more processor 21 may further be used to determine compliance with the dental plan. As previously mentioned, compliance with the dental plan may be determined based on strict or less than strict adherence to one or more aspect of the dental plan. Data corresponding to the compliance determination may be stored in the memory 25 of the case 100.

In some embodiments the data corresponding to the compliance determination may be a binary indication. That is, the data may indicate only whether compliance has been met, essentially a yes or no indication. In other embodiments, data corresponding to the adherence to the dental plan may be sent by the case 100. In other embodiments, a binary indication as to adherence may be sent by the case 100.

The external server 400 and/or the client may request further information from the case 100 based on the received compliance determination and/or adherence determination. For instance, the external server 400 and/or client may request further information when the received compliance determination indicates non-compliance with the prescribed treatment plan. Such further information may include the adherence determination and/or level of adherence to various aspects of the dental plan.

As another example, the case 100 may send additional information corresponding to adherence to the dental plan without receiving a request from the external server 400 and/or the client. For instance, the case 100 may send the additional information in a binary fashion if adherence within a threshold has been met, and may send the level of adherence if the threshold has not been met.

Additionally, the case 100 may only send information corresponding to adherence to various aspects of the dental plan if compliance has not been met. For example, a simple binary indication as to compliance can be sent if compliance has been determined. However, if non-compliance has been determined, the case 100 may send information concerning adherence to the various aspects of the dental plan. As mentioned above, the information concerning adherence to one or more various aspects of the dental plan may each be transmitted in binary and/or analog (level of adherence) fashion. Also, the binary non-compliance determination may also be sent along with the information concerning adherence.

In another embodiment, the compliance determination may be made by the external server 400 and/or the client. In such an embodiment, the case 100 may transmit data corresponding to adherence or sensor(s) reading(s) to the external server 400 and/or client. Based on such received data, the external server 400 and/or client may determine compliance, in a similar fashion as that which has been previously described.

There are various benefits to dynamically or statically selecting which device processes the data for determining compliance and/or adherence to aspects of the dental plan. For instance, one such benefit is more beneficial power management. For example, a large portion of the power utilization of the case 100 might be due to the functioning of the communication array 23. In such a situation, it may be more beneficial to reduce the amount and/or frequency of data that needs to be transmitted by the communication array 23. This could potentially be done by performing the compliance determination by the one or more processor 21 of the case 100, and only sending data corresponding to a binary compliance determination. To implement further battery management, the frequency of transmitting such a determination may be reduced. For instance, a weekly aggregate of the compliance determinations may be sent, instead of daily compliance determination.

However, if the communication array 23 is acceptably power efficient or additional power management is desired, it may necessary to reduce the amount power expended by the one or more processor 21 of the case 100. For example, this may be accomplished by the amount of processing required by the one or more processor 21. In one situation, the processing requirement of the one or more processor 21 may be reduced by not performing the compliance determination and/or the adherence determination. In such a situation, the necessary data may be transmitted to the client and/or external server 400 for processing the determination.

In some embodiments, the way of determining compliance to a dental plan and/or adherence to an aspect of the dental plan may be dynamically adjusted based on data corresponding to previous determinations of compliance and/or based on current or pervious data corresponding to adherence to one or more aspect of the dental plan. Such data corresponding to adherence does not need to be, but may in certain situations, used for determining compliance. For instance, the aspect of pain resulting from use of the dental appliance may be used for dynamically adjusting the way of determining compliance. For example, if the data corresponding to pain indicates that pain is tolerable at a point during the treatment process, the previously required level of adherence to aspects of the dental plan, such as wear time, may be adjusted. In one situation, adherence to the aspect of wear time may be determined based on a lower time period requirement then previously needed, or compliance may no longer take into account the aspect of wear time at all.

However, in certain situations, the way of determining compliance to a dental plan and/or adherence to an aspect of the dental plan may be adjusted without somehow notifying or implying such an adjustment to the patient, client, and/or server 400. For instance, there may be an indication of non-compliance provided to a patient. However, the data sent to the client and/or external server 400 may indicate that compliance has actually been determined. Benefits of dynamically adjusting the compliance determination and/or adherence determination include, but are not limited to, more tailored treatment plan and/or more accurately updated or newly provided dental plans and/or appliances. Further benefits include, but are not limited to, encouraging the patient to maintain a good habit of striving for compliance and/or adherence without negatively affecting treatment time, progress, or any decisions as to an updated or newly created treatment plan and/or appliance.

As discussed above, compliance with a dental treatment plan may be determined based on an aggregate of numerous compliance determinations. Based on the aggregate compliance determination and/or level of compliance, a new or updated treatment plan and/or dental appliance may be provided to the patient. The updated treatment plan may be transmitted from the external client 400 to the client and/or case 100. Alternatively, the updated treatment plan may be transmitted from the client to the case 100.

The updated treatment plan and/or dental appliance may be based not only on the aggregate or level of compliance, but may also be based on the aspects of the dental plan that have been adhered to and/or to what level. For instance, if the aspect of the length of dental appliance 200 wear time per day is deemed to be adhered to, but the aspect of cleanliness of the dental appliance 200 is determined to not have been adhered to, then the dental treatment plan may be updated such that compliance is not just based on wear time, but also requires a certain level of cleanliness. As another example, if the aspect of cleanliness has not been determined to be adhered to, then an updated dental appliance may be provided to the patient that is made of a different material or has other features to aid in cleanliness. As yet another example, if the aspect of the structural integrity of the dental appliance 200 has been determined to not adhere with the dental treatment plan, then an updated dental appliance may be provided. For instance, the updated dental appliance may be made to have additional structural integrity or a different structural form to compensate of such determination of a lack of adherence to the structural integrity.

An updated dental treatment plan and/or appliance may be provided at regular intervals or based on a predetermined schedule. Alternatively, an updated dental treatment plan and/or appliance may be provided on an as needed basis based on the compliance determinations and/or adherence determinations. As yet another example, an updated dental treatment plan and/or appliance may be provided based on a single or aggregate determination of the compliance or adherence. For instance, if the aspect of the structural integrity of the dental appliance is determined not to be adhered to by a certain amount, then an updated dental appliance may be provided to the patient earlier than originally scheduled or on an as needed basis.

The data corresponding to compliance and/or adherence may be sent to and/or reviewed by a medical practitioner. Based on the medical practitioner's review and analysis, the medical practitioner may provide an updated or new treatment plan and/or an updated or new medical device, such as a dental appliance. The new or updated treatment plan and/or medical device may be sent to the patient, the external server 400, the client, the case 100, or any combination thereof. If the new or updated treatment plan is provided only to the external server 400, the external server 400 may forward such a treatment plan to the client directly, the case 100 directly, or the case 100 via the client. Alternatively, the client and/or external server 400 may determine the new or updated treatment plan and/or dental appliance without input from a medical practitioner.

In some embodiments, data corresponding to determination(s) of compliance and/or adherence to aspects of a dental plan may be used with data corresponding to determination(s) of compliance and/or adherence to aspects of other dental plans. For instance, data corresponding to a current dental plan may be combined with or compared to data corresponding to one or more previous dental plan. In such a situation, the combined or compared data may be used to develop a new or updated dental plan and/or appliance, if necessary.

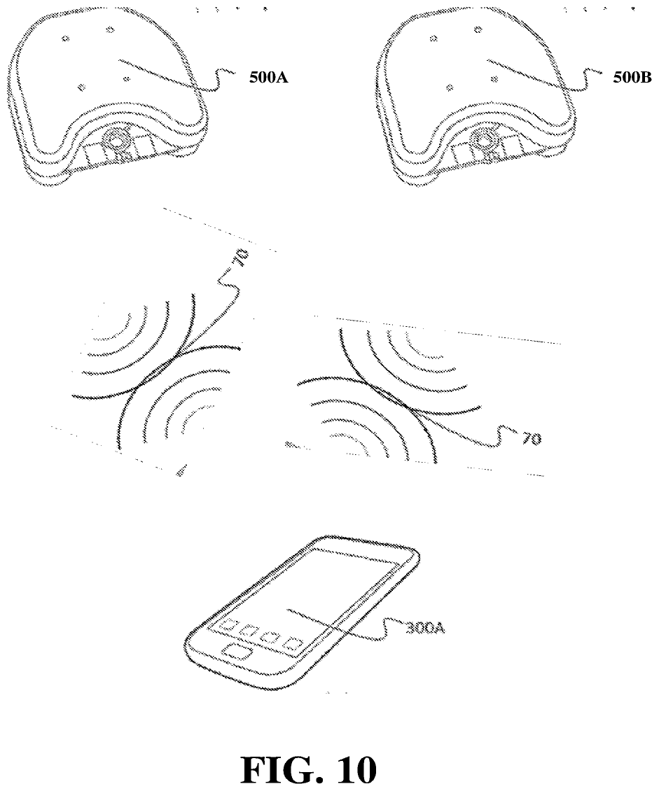

In some embodiments, a patient may use more than one case, for example cases 500A and 500B of FIG. 10, to store the dental appliance 200 at various times during the day. This may be desirable because the patient may prefer to keep one case at home and one case in their bag. In such a situation, the information collected by each of the two cases 500A, 500B should be synchronized to ensure a proper determination of compliance. For instance, the client, such as the smart phone 300A and/or computer 300B, and/or external server 400 could function as a centralized device for synchronizing the information collected by the two cases 500A, 500B. Such synchronization could occur by concurrently communicating with the two cases 500A, 500B, or could occur by independently communicating with each of the two cases 500A, 500B.

The synchronized data collected by the client and/or external server 400 may include an aggregation of the data sent by each of the cases 500A, 500B. As previously mentioned, the data sent by a case may merely include a binary compliance determination, or may alternatively or in addition to, include information relating to adherence, whether strict or less than strict, to different aspects of the dental plan. Which information is to be sent by the dental appliance case may depend on the aspect of the dental plan of which adherence is being determined, and if such determinations could be or have been detected by a single case. For instance, if the aspect of wear time is the only desirable parameter or only what is needed to determine compliance, then only a compliance determination may be transmitted by the case if the determination indicated non-compliance. However, if it was determined that the wear time would adhere to the dental plan, thereby indicating compliance, the case may send data corresponding to such an aspect to the client or the external server 400. This may be done because the aggregate time the dental appliance is detected in all of the cases may result in a non-compliant determination. As another example, if the aspect of cleanliness is being determined for adherence, the data from only a single case may be necessary to be sent to the client or external server.

Depending on which device is performing the compliance determination, the synchronized and/or aggregate data may be entirely stored on the client and/or external server. Alternatively, the data may first be collected and stored on the client and/or external server, and may subsequently be transmitted to the device that is performing the determination, such as one of the two cases 500A, 500B. As another example, the cases 500A, 500B may be in direct communication with each other such that one or both of the cases 500A, 500B stores the synchronized and/or aggregated data, and one or both of the cases 500A, 500B can perform the compliance determination.

In some embodiments, the type and amount of data sent by the case may be dynamically adjusted. For instance, the case may send different data depending on whether the patient intends to use one or more than one case to temporarily store the dental appliance 200. For instance, the case may only send a compliance determination if only one case is to be used by the patient, and may alternatively send the level of adherence to aspects of the dental plan when more than one case is to be used by the patient. The amount and type of data may also be adjusted in circumstances where communication with the client and/or external server 400 is undesirable or in order to implement power management protocols.

In some embodiments, a treatment plan aspect of the structural integrity of a dental appliance may be determined based on a total wear time of the dental appliance. For instance, a certain total wear time may indicate that the structural integrity of the dental appliance is insufficient to properly perform the desired treatment. In such a case, a new dental appliance may be sent to the patient when the patient has, or is indicated to soon have, based on previously obtained data corresponding to wear time, worn the current dental appliance for a certain amount of time. As indicated above, determination of adherence to aspects of a dental plan may be transmitted to a client and/or external server. Such information may be used by dentists or dental appliance companies for ensuring that the patient is utilizing an appropriate dental appliance and for providing the patient an appropriate new dental appliance if the current dental appliance is deemed to currently or soon have inadequate structural integrity. In some situations, this may be useful when the current dental treatment plan has the purpose of maintaining a certain alignment, within a degree of error, rather than attempting to shift the teeth to a new location.

In some embodiments, the total wear time alone may not be sufficient to determine if a new dental appliance should be sent to the patient. For example, the total wear time may also be supplemented with, or replaced by, information such as the length of time the patient is not wearing the dental appliance, the average wear time over a fixed period of time, the consistency of wearing the dental appliance, the amount of pain experienced by the patient, fit, etc., or any combination thereof. For instance, an updated or new dental appliance and/or plan may be provided if the patient has not, or for an insufficient time, worn the current dental appliance. In such a situation, it may be anticipated that at least portions of one or more previous dental plan may need to be repeated or an entirely new dental plan and/or appliance will need to be developed. As such, an updated and/or new dental appliance or plan may be provided earlier than originally planned, such as originally planned based on the structural integrity. The level of pain experienced by the patient may also indicate an issue with the current dental plan and/or appliance. For example, an increased indication of pain experienced by the patient may indicate that the teeth have become misaligned and further alignment may be needed. In such a situation, an updated or new dental appliance and/or plan may be provided.

Any or all of the information, data, and/or determination(s) may be sent to and/or stored in one or more external server, whether such an external server is the above mentioned external server 400 or a separate external server. Such information, data, and/or determination(s) may be analyzed separately from and/or aggregated with information, data, and/or determination(s) from that previously received for that patient and/or other patients, and/or other information or data for the purpose of creating more desirable dental treatment plans for current and/or future patients. For instance, an analysis of the information, data, and/or determination(s) may indicate that total treatment time may be shorter than originally thought. This may be especially true for patients that have a higher pain threshold and/or there is an indication that the perceived level of pain has reduced sooner than expected. The analysis may further indicate that the amount of dental appliance wear time may be less per fixed period of time than originally thought. Combination(s) of parameters may also be analyzed to determine more suitable dental plans and/or appliances.

For example, an analysis of the information, data, and/or determination(s) may indicate that a patient's pain threshold and/or perception of pain changes over time. For instance, the patient may become increasingly desensitized to pain over a period of a single treatment plan or over numerous treatment plans. In such a situation, a treatment plan(s) approach may be made where the teeth are moved in a dynamic fashion, such as increasing the movement speed at later points in the treatment process. Accordingly, the patient may perceive the same or less pain throughout treatment, all while reducing the overall required treatment time and/or speeding up aspects of the treatment process.

As another example, an analysis of the information, data, and/or determination(s) may indicate that if a dental appliance progresses treatment quicker, even though it may result in greater pain to the patient, the amount of wear time per fixed period of time may be reduced, while still maintaining a suitable treatment progression.

As yet another example, an analysis of the information, data, and/or determination(s) may indicate that a dental appliance may be worn less per fixed period of time if the length of one or more treatment plan is increased.

As still another example, an analysis of the information, data, and/or determination(s) may indicate the level that various personal and/or psychological traits affect a treatment. For instance, the information, data, and/or determination(s) may be used separately from or added to other data collected about the patient to individualize treatment plans for other patient with similar personal or psychological traits. For example, age and/or gender may be determined to suggest the perceived level of pain of a patient, thereby suggesting that a treatment plan could be different for that patient than for someone of a different age and/or gender. Another example, the geographical location of a patient may indicate the level of compliance to a treatment plan. Such geographical location may be based on only a single individual, or based on the relative location to other patients undergoing treatment. Yet another example may indicate the level of conditioning or habit forming that is needed in order to suggest that a patient will obtain and maintain an appropriate level of compliance to a dental plan.

All of the above analyses have the benefit of improving the treatment process for patients. Not only are more accurate treatment plans able to be provided, but other benefits include, but are not limited to, allowing a patient to custom tailor a treatment plan to their preferences and life style.

A number of examples have been described above. Nevertheless, it will be understood that various modifications may be made. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the following claims.

1. A method of determining compliance with a treatment plan, comprising the steps of:
parsing one or more aspect of a treatment plan;
sensing one or more physical feature of a medical device using one or more sensor separately provided from the medical device, the sensed feature(s) corresponding to the one or more aspect of the treatment plan;
determining adherence to each parsed aspect of the treatment plan based on the sensed feature(s) of the medical device;
determining compliance with the treatment plan based on the determination(s) of adherence of the parsed aspect(s) of the treatment plan.

2. The method of claim 1, wherein the aspect of the treatment plan corresponds to a wear time of the medical device.

3. The method of claim 2, wherein compliance has been determined to have been met when the wear time of the medical device has met or exceeded a threshold.

4. The method of claim 1, wherein the sensed feature of the medical device is an amount of time the medical device is located within a storage cavity.

5. The method of claim 1, wherein compliance is determined to have been met when adherence is determined to be less than strict adherence.

6. The method of claim 1, wherein compliance is determined to have been met when adherence is determined to be strict adherence to one aspect and strict or less than strict adherence to the other aspect.

7. The method of claim 1, further comprising the step of updating the treatment plan when compliance is determined to not have been met.

8. The method of claim 1, further comprising the step of modifying how compliance is determined based on previous compliance determinations.

9. The method of claim 1, further comprising the step of providing a new or updated medical device based on the compliance determination.

10. The method of claim 1, further comprising the step of transmitting the compliance determination to an external device.

11. The method of claim 1, further comprising the step of transmitting an aggregate compliance determination to an external device, wherein the aggregate compliance determination corresponds to a plurality of compliance determinations.

12. The method of claim 1, further comprising transmitting the adherence determination when compliance has been determined to not have been met.

13. The method of claim 1, further comprising transmitting data corresponding to the sensed one or more feature of the medical device when compliance has been determined to not have been met.
